(19) United States
(12) Patent Application Publication
(10) Pub. No.: US 2025/0325805 A1
(43) Pub. Date: Oct. 23, 2025

(54) SUPPORT DEVICE WITH EARTHING THERAPY DEVICE

(71) Applicant: Earthlite, LLC, Vista, CA (US)

(72) Inventors: James Chenevey, Encinitas, CA (US); Garth Weber, Fallbrook, CA (US)

(21) Appl. No.: 19/184,620

(22) Filed: Apr. 21, 2025

Related U.S. Application Data
(60) Provisional application No. 63/637,357, filed on Apr. 22, 2024.
(60) Provisional application No. 63/637,236, filed on Apr. 22, 2024.

(51) Int. Cl. A61N 1/14; A61H 37/00

(52) U.S. Cl. CPC A61N 1/14; A61H 37/00; A61H 2201/0138; A61H 2201/0149; A61H 2201/10

(57) ABSTRACT

A support device assembly (11) for supporting a user (10) relative to a surface (18) includes a support device (12) and an earthing therapy device (20). The support device (12) supports the user (10) relative to the surface (18). The support device (12) includes a device body (14). The earthing therapy device (20) is configured to provide grounding therapy to the user (10) as the user (10) is positioned on the support device (12). The earthing therapy device (20) is one of (i) incorporated within, and (ii) coupled to the device body (14). The support device (12) can be a treatment device that supports the user (10) relative to the surface (18) as the user (10) is receiving a therapeutic treatment.

### RELATED APPLICATIONS

This Application claims priority on U.S. Provisional Patent Application Ser. No. 63/637,236 filed on Apr. 22, 2024, and entitled “EARTHING STRIP FOR INCORPORATION WITHIN A SUPPORT DEVICE”; and U.S. Provisional Patent Application Ser. No. 63/637,357 filed on Apr. 22, 2024, and entitled “GROUNDED ATTACHMENT POINTS FOR EARTHING THERAPY DEVICE”. To the extent permitted, the contents of U.S. Provisional Patent Application Ser. Nos. 63/637,236 and 63/637,357 are incorporated in their entirety herein by reference.

### BACKGROUND

As the benefits of therapeutic treatments such as therapeutic massage and/or other related treatments are becoming more widely appreciated, more and more people are participating in such therapeutic treatments. It is common for people to enjoy resting comfortably while they receive one or more therapeutic treatments. A typical support device can be a treatment device, such as a massage table or a massage chair, that allows the patient to be in a comfortable resting position while receiving a therapeutic treatment, such as a massage.

Additionally, many people subscribe to the health benefits of having a connection to a grounding source. Grounding, or earthing, is a therapeutic technique that involves doing activities that “ground” or electrically reconnect the recipient to the earth. This practice relies on earthing science and grounding physics to explain how electrical charges from the earth can have positive effects on your body. The dissipation of charge in such practice is thought to promote health.

Indeed, many people actively practice grounding themselves. Insulated devices such as grounding pads, straps, blankets, and sheets have commonly been used to provide earthing therapy. To aid in the ability of grounding people utilizing an insulated device, a conductive surface is needed, and that conductive surface must have a path to ground.

For example, one typical method for providing the desired earthing therapy is to use a grounding pad that is typically placed on a surface, such as a floor or the ground. The users can then place their feet on the grounding pad to discharge any built-up voltage. The grounding pad consists of a reasonably conductive surface with a grounding wire attached, which is, in turn, attached to a grounding source, often through the ground pin on an electrical outlet. Such method typically involves the use of grounding wires that would run to a wall outlet for their grounding source, externally from the grounding pad. Unfortunately, this can create a dangerous tripping hazard.

Other attempts to provide grounding or earthing products have not been fully successful, often suffering from being too expensive, having a relatively short usage life, and/or being inconvenient for use with multiple treatment recipients.

Accordingly, it is desired to provide a safe, convenient and effective manner in which a user can receive both a therapeutic treatment, such as a therapeutic massage, and an earthing therapy treatment.

### SUMMARY

The present invention is directed toward a support device assembly for supporting a user relative to a surface. In various embodiments, the support device assembly includes a support device and an earthing therapy device. The support device supports the user relative to the surface. The support device includes a device body. The earthing therapy device is configured to provide grounding therapy to the user as the user is positioned on the support device. The earthing therapy device is one of (i) incorporated within, and (ii) coupled to the device body.

In many embodiments, the earthing therapy device includes a conductive surface that is formed from at least one of conductive leather, conductive vinyl, conductive plastic, and conductive cloth.

In some embodiments, the earthing therapy device includes a conductive surface that is incorporated into the device body.

In other embodiments, the earthing therapy device includes a conductive surface that is coupled to the device body.

In certain embodiments, the device body includes a body cover. In some embodiments, the earthing therapy device is one of incorporated into and coupled to the body cover.

In some embodiments, the earthing therapy device is one of incorporated into and coupled to an outer surface of the body cover.

In certain embodiments, the earthing therapy device is one of incorporated into and coupled to an inner surface of the body cover.

In some embodiments, the earthing therapy device is coupled to the inner surface of the body cover using conductive thread.

In certain embodiments, the conductive thread is formed from at least one of silver, copper, nickel, tin, and stainless steel.

In some embodiments, the support device assembly further includes a comfort enhancer that is configured to be selectively positioned adjacent to the device body, and the earthing therapy device includes a conductive surface that is positioned at least partially about the comfort enhancer.

In certain embodiments, the earthing therapy device is configured to couple the comfort enhancer to the device body.

In many embodiments, the support device assembly further includes a connection assembly that selectively electrically couples the earthing therapy device to a grounding source.

In some embodiments, the connection assembly includes a grounding wire having a first end that is selectively electrically coupled to the earthing therapy device, and a second end that is selectively electrically coupled to the grounding source.

In some embodiments, the first end of the grounding wire is selectively electrically coupled to the earthing therapy device via a surface connector, the surface connector including a first connector member that is connected to the earthing therapy device, and a second connector member that is connected to the grounding wire near the first end of the grounding wire, the second connector member being configured to selectively engage the first connector member.

In certain embodiments, the first connector member is a snap button, and the second connector member is a button insert that is configured to selectively engage the snap button.

In some embodiments, the second end of the grounding wire is selectively electrically coupled to the grounding source via a source connector, the source connector including a first connector member that is connected to the grounding source, and a second connector member that is connected to the grounding wire near the second end of the grounding wire, the second connector member being configured to selectively engage the first connector member.

In certain embodiments, the grounding source includes a wall outlet.

In some embodiments, the first connector member includes a power strip that is selectively electrically coupled to the grounding source, and the second connector member includes a ground adapter that is connected to the grounding wire near the second end of the grounding wire, the ground adapter being configured to selectively electrically engage the power strip.

In certain embodiments, the first connector member is incorporated within the device body.

In some embodiments, the device body of the support device further includes a body frame, and the first connector member includes a member receiver that is incorporated within the body frame, and the second connector member includes a grounding lug that is configured to selectively engage the member receiver.

In many embodiments, the support device is a treatment device that supports the user relative to the surface as the user is receiving a therapeutic treatment.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified perspective view illustration of a user and an embodiment of a support device assembly having features of the present invention, the support device assembly including a support device having a device body, and an earthing therapy device that is incorporated within the device body;

FIG. 2A is a simplified perspective view illustration of an embodiment of the device body with the earthing therapy device incorporated therein, and a connection assembly that is configured to be selectively electrically coupled to the earthing therapy device and a grounding source;

FIG. 2B is an enlarged perspective view illustration of a portion of the device body and the connection assembly as defined by circle B-B in FIG. 2A;

FIG. 3A is a simplified perspective view illustration of another embodiment of the device body with the earthing therapy device incorporated therein, and another embodiment of the connection assembly that is configured to be selectively electrically coupled to the earthing therapy device and the grounding source;

FIG. 3B is an enlarged perspective view illustration of a portion of the device body and the connection assembly as defined by circle B-B in FIG. 3A;

FIG. 4 is a simplified perspective view illustration of still another embodiment of the device body with the earthing therapy device incorporated therein, and still another embodiment of the connection assembly that is configured to be selectively electrically coupled to the earthing therapy device and the grounding source;

FIGS. 5A-5F are alternative embodiments for the size, shape and positioning of the earthing therapy device as the earthing therapy device is incorporated within a body cover of the device body;

FIG. 6 is a simplified partially exploded perspective view illustration of another embodiment of a support device assembly having features of the present invention, the support device assembly including another embodiment of the support device, the earthing therapy device, and the connection assembly that effectively couples the earthing therapy device to the support device, while providing a conductive path and a grounding point for the earthing therapy device;

FIG. 7 is a simplified perspective view illustration of a portion of still another embodiment of the support device assembly having features of the present invention;

FIG. 8A is a simplified top view illustration of an embodiment of the earthing therapy device and a portion of an embodiment of the connection assembly illustrated in FIG. 6;

FIG. 8B is a simplified end view illustration of the earthing therapy device and the connection assembly illustrated in FIG. 8A;

FIG. 8C is a simplified side view illustration of the earthing therapy device and the connection assembly illustrated in FIG. 8A;

FIG. 8D is an enlarged schematic view illustration of a portion of the earthing therapy device as defined by circle D-D in FIG. 8C; and

FIG. 9 is a simplified perspective view illustration of a portion of still yet another embodiment of the support device assembly having features of the present invention.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of an earthing therapy device, which can be used in conjunction with a support device that is configured to support a user relative to a surface. In particular, in various embodiments, the present invention is directed toward a support device with an earthing therapy device being incorporated therein and/or coupled thereto. As referred to herein, the earthing therapy device being “coupled” to the support device is intended to signify that there is a close physical and/or mechanical linking or fastening of the earthing therapy device to the support device.

The earthing therapy device can be electrically coupled to a grounding source through use of a connection assembly. In some embodiments, the support device includes a device body, and the earthing therapy device can be provided in the form of a conductive material and/or a conductive surface that is incorporated into the device body. In other embodiments, the support device includes a device body that provides and/or includes grounded attachment points so that an earthing therapy device, such as in the form of a conductive band and/or a conductive surface, can be selectively coupled thereto. In any such embodiments, the user is able to conveniently receive an earthing therapy treatment at the same time that the user is being supported by the support device relative to the surface.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

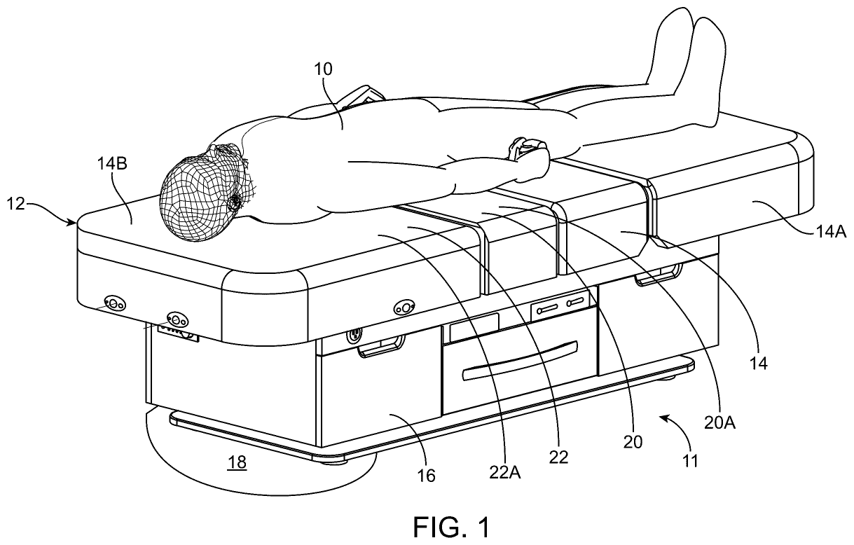

FIG. 1 is a simplified perspective view illustration of a user 10 and an embodiment of a support device assembly 11 having features of the present invention that is configured to support the user 10 relative to a surface 18, such as a floor or the ground.

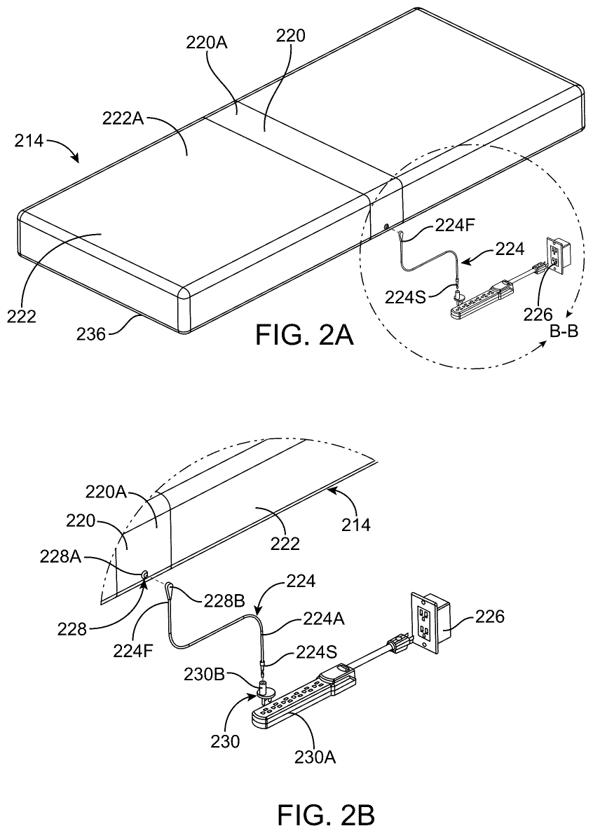

As shown, the support device assembly 11 includes a support device 12, and an earthing therapy device 20 that is coupled to and/or incorporated within the support device 12. In many embodiments, the support device assembly 11 further includes a connection assembly 224 (illustrated in FIG. 2A) that is configured to selectively electrically couple the earthing therapy device 20 to a grounding source 226 (illustrated in FIG. 2A), such as a wall outlet, or another type of ground.

The design of the support device 12 can be varied to suit the needs of the user 10. As illustrated in the embodiment shown in FIG. 1, the support device 12 includes (i) a device body 14 that is configured to support the user 10 of the support device 12, (ii) a support assembly 16 that supports the device body 14 above and/or relative to the surface 18, and (iii) the earthing therapy device 20 that is coupled to and/or incorporated within the device body 14. In many embodiments, such as shown in FIG. 1, the earthing therapy device 20 can be provided in the form of a conductive material and/or a conductive surface that is coupled to and/or incorporated within the device body 14. Alternatively, the earthing therapy device 20 can be provided in another suitable form. Still alternatively, the support device 12 can include more components than those specifically illustrated in FIG. 1. For example, in one non-exclusive alternative embodiment, the support device 12 can further include a headrest assembly (not shown) that is configured to support a face or head of the user 10 when the user 10 is positioned on the device body 14. Such headrest assembly can alternatively be coupled to the device body 14 or integrally formed into the device body 14.

As an overview, the support device 12 is configured to support the user 10 above and/or relative to the surface 18 as the user 10 is resting, relaxing and/or receiving a massage or other therapeutic treatment. Additionally, the earthing therapy device 20 is configured to provide desired grounding therapy to the user 10 as the user 10 is supported on the support device 12. The earthing therapy device 20 that is incorporated into and/or coupled to the device body 14 of the support device 12 is selectively electrically coupled to the grounding source 226 through use of the connection assembly 224. More specifically, as illustrated and described in greater detail herein below, the connection assembly 224 includes a first end 224F (illustrated in FIG. 2A) that is selectively electrically coupled to the earthing therapy device 20, and a second end 224S (illustrated in FIG. 2A) that is selectively electrically coupled to the grounding source 226.

As illustrated in FIG. 1, the support device 12 is shown as a treatment device in the form of a massage table that supports the user 10 relative to the surface 18, with the user 10 lying down while receiving a therapeutic treatment such as a massage. However, it is appreciated that the support device 12 can have another suitable design, and does not have to be provided in the form of a massage table. For example, in one non-exclusive alternative embodiment, the support device 12 can be provided as a treatment device in the form of a massage chair that supports the user 10 in a reclined and/or seated position relative to the surface 18 while receiving a therapeutic treatment such as a massage. Still alternatively, the support device 12 can have another suitable form for purposes of supporting the user 10 relative to the surface 18. For example, it is appreciated that the support device 12 can be another type of support device, such as a bed, a couch, a chair, a lounger, etc. More particularly, it is appreciated that the support device 12 having features of the present invention is usable to provide an earthing therapy treatment in various non-exclusive implementations, such as by private citizens in their home furnishings (beds, couches, chairs, etc.), by hotels in their guest rooms (beds, couches, chairs, etc.), and/or in spa environments (massage tables, massage chairs, loungers, etc.).

In the embodiment shown in FIG. 1, the device body 14 includes a plurality of body sections 14A that are movably coupled to one another such that an upper surface 14B of the support device 12 and/or the device body 14 can be adjusted to improve the comfort for the user 10 of the support device 12. In particular, in this embodiment, the device body 14 includes four body sections 14A that are movably coupled to one another so as to enable the adjustment of a height and/or an angle of the head, the torso and/or the legs of the user 10 relative to other body parts, regardless of the size of the user 10. Alternatively, the device body 14 can be formed with only a single body section.

As shown in this embodiment, the device body 14 is substantially rectangle-shaped. Alternatively, the device body 14 can have another suitable shape. For example, in certain non-exclusive alternative embodiments, the device body 14 can be substantially oval-shaped, oblong-shaped, rectangular-shaped with one or more rounded corners, or another suitable shape.

In some embodiments, the device body 14 can include a body frame (not shown), a pad (not shown), and a body cover 22. Alternatively, for example, the device body 14 can be made without the pad and/or without the body cover 22.

The body frame supports the user 10 when the user 10 is positioned on the support device 12. The body frame is generally rigid and can be made of a rigid material such as wood, aluminum, steel, plastic or other suitable materials. In alternative, non-exclusive embodiments, the body frame has a thickness of between approximately five millimeters and 30 millimeters. More particularly, the body frame can have a thickness of approximately 5, 6, 7, 8, 9, 10, 11, 12, 13, 14, 15, 16, 17, 18, 19, 20, 21, 22, 23, 24, 25, 26, 27, 28, 29, or 30 millimeters. However, alternatively, the body frame can have a thickness that is greater than or less than the noted thicknesses.

The pad is secured to and can be positioned substantially on top of the body frame. The pad provides a cushion for the comfort of the user 10 resting on the support device 12. Non-exclusive examples of suitable materials for the pad include foam, memory foam, fleece pads, etc. The pad can have any suitable thickness and any suitable stiffness/resiliency for purposes of comfortably supporting users 10 of many different sizes.

The body cover 22 secures the pad to the body frame and provides a protective covering for the pad. Non-exclusive examples of suitable materials for the body cover 22 include leather, vinyl, plastic, and cloth.

As noted above, the support assembly 16 supports the device body 14 above and/or relative to the surface 18. More particularly, as shown in FIG. 1, the support assembly 16 extends between the device body 14 and the surface 18 to maintain the device body 14 positioned above and away from the surface 18. It is appreciated that the support assembly 16 can have any suitable design for purposes of supporting the device body 14 above and/or relative to the surface 18. For example, in one embodiment, the support assembly 16 can include one or more legs and/or feet that are fairly lightweight, but still provide desired stability for the support device 12. Alternatively, the support assembly 16 can have another suitable design.

In some embodiments, the support assembly 16 can be adjustable so as to enable a height of the device body 14 relative to the surface 18 to be selectively adjusted. For example, in one non-exclusive embodiment, the support assembly 16 can have legs that include individual leg members that are telescopingly coupled to one another to enable the selective height adjustment. Alternatively, the support assembly 16 can have another suitable design for purposes of enabling the desired selective height adjustment. It is appreciated that, in different embodiments, any such height adjustment can be accomplished manually or in an automated manner.

In certain embodiments, the support assembly 16 can be further configured to provide one or more storage compartments, such as drawers, that are usable for storage of materials that can be used during any therapeutic treatments.

The earthing therapy device 20, such as a conductive material and/or a conductive surface 20A in certain non-exclusive embodiments, is configured to provide desired grounding therapy or earthing therapy to the user 10 as the user 10 is supported on the support device 12. In various embodiments, the earthing therapy device 20 is coupled to and/or incorporated within the body cover 22 of the device body 14. For example, the earthing therapy device 20 can be fixedly secured to and/or integrally incorporated within the body cover 22 of the device body 14. Alternative, in other embodiments, the earthing therapy device 20 can be coupled to and/or incorporated within another portion of the device body 14, such as being coupled to and/or incorporated within the pad of the device body 14.

The earthing therapy device 20 and/or the conductive surface 20A can be formed from and/or can include any suitable conductive materials. For example, in certain non-exclusive alternative embodiments, the earthing therapy device 20 and/or the conductive surface 20A can be formed from and/or can include one or more of conductive leather, conductive vinyl, conductive plastic, and conductive cloth, which can be similar to the materials used for the body cover 22, with the exception of including conductive properties. Alternatively, the earthing therapy device 20 and/or the conductive surface 20A can be formed from and/or can include any other suitable conductive materials.

In the case of massage tables, the earthing therapy device 20 could be sewn into the pad, or the body cover 22, and would still be conductive with a thin sheet over it. It is appreciated that this can be a particularly valuable feature as there is no special sheet required.

In different embodiments, as shown in greater detail in subsequent Figures, the earthing therapy device 20 can be incorporated into the device body 14 in different manners. In certain embodiments, the earthing therapy device 20 is sewn into the body cover 22 of the support device 12 where the user 10 is most likely to come in contact with it. In some embodiments, the earthing therapy device 20 could be a simple thin strip of conductive material attached as part of the body cover 22 of the device body 14 or up to essentially the entirety of the device body 14. The earthing therapy device 20, or a conductive surface 20A thereof, is attached to the grounding source 226, via the connection assembly 224, such as a grounding wire in some non-exclusive embodiments. As noted, in many embodiments, the grounding source 226 can be a wall outlet. Alternatively, the support device assembly 11 and/or the earthing therapy device 20 can use another suitable grounding source 226, which in some non-exclusive alternative embodiments can incorporate a ground line or a metal frame tied to ground. For example, in one non-exclusive alternative embodiment, the grounding source 226 can be provided as or integrated within the body frame of the device body 14.

As illustrated in this embodiment, since the earthing therapy device 20 is effectively incorporated as part of the support device 12, there is no setup or storage needed, and the conductive surface 20A of the earthing therapy device 20 is always available for dissipation of voltage. For example, one iteration of the present invention entails sewing the earthing therapy device 20 into the body cover 22 such that the conductive surface 20A is part of an outer surface 22A of the body cover 22. Another iteration of the present invention entails sewing the earthing therapy device 20 to the underside of the body cover 22 using conductive thread. Still another iteration of the present invention entails sewing or otherwise incorporating the earthing therapy device 20 into the pad of the device body 14.

FIG. 2A is a simplified perspective view illustration of an embodiment of the device body 214 with the earthing therapy device 220 incorporated therein, and a connection assembly 224 that is configured to provide desired selective electrically coupling between the earthing therapy device 220 and a grounding source 226, such as a wall outlet, or another type of ground. More particularly, in this embodiment, the earthing therapy device 220 is provided in the form of a conductive fabric and/or a conductive surface 220A that is incorporated into the device body 214, such as into the body cover 222 of the device body 214. Additionally, as shown, the connection assembly 224 includes a first end 224F that is configured to be selectively electrically coupled to the earthing therapy device 220, and a second end 224S that is configured to be selectively electrically coupled to the grounding source 226.

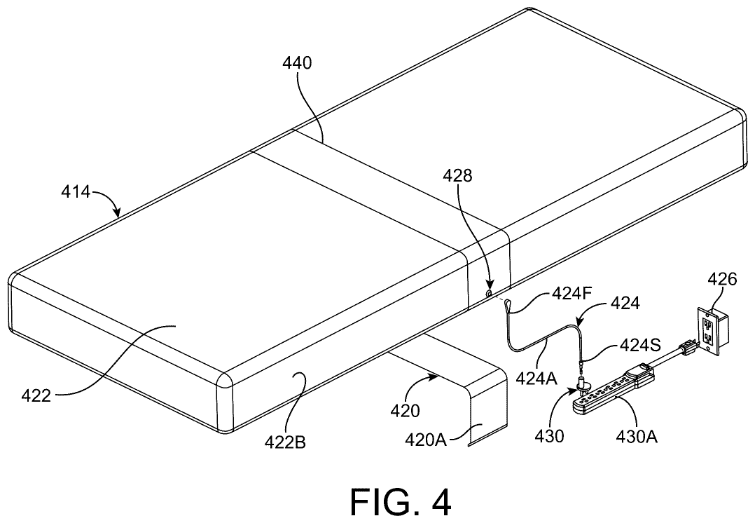

The earthing therapy device 220 and/or the conductive surface 220A can be incorporated into the body cover 222 of the device body 214 in any suitable manner and in any suitable position. In the embodiment shown in FIG. 2A, the earthing therapy device 220 and/or the conductive surface 220A is sewn onto or otherwise attached to an outer surface 222A of the body cover 222. Alternatively, the earthing therapy device 220 and/or the conductive surface 220A can be incorporated into the body cover 222 in another suitable manner. For example, in one non-exclusive alternative embodiment, the earthing therapy device 220 and/or the conductive surface 220A can be sewn into an inner surface (not shown) or the underside of the body cover 222 using conductive thread, such as shown in FIG. 4.

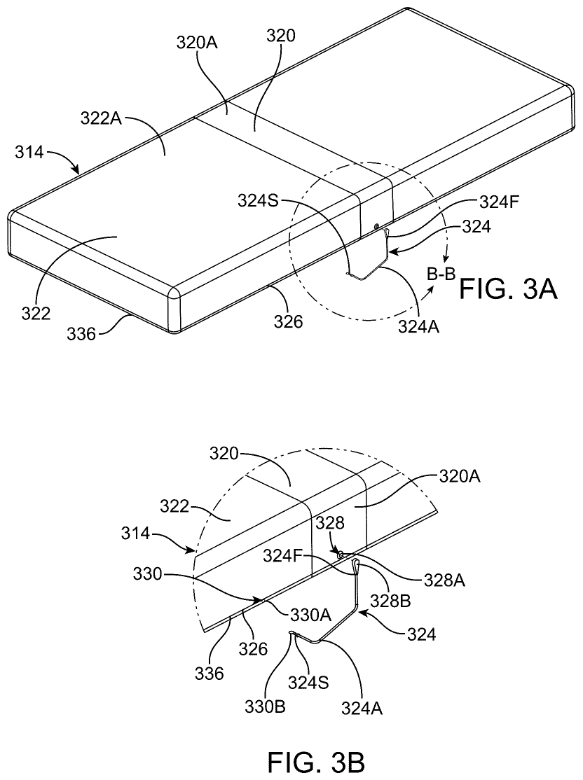

The design of the connection assembly 224 can be varied. As shown in FIG. 2A, the connection assembly 224 is electrically coupled to and between the earthing therapy device 220 and/or the conductive surface 220A and the grounding source 226. As noted, in some embodiments, the grounding source 226 can be provided via a wall outlet, such as via the grounding pin of a wall outlet. Alternatively, the grounding source 226 can be provided via a body frame 236 of the device body 214, such as shown in FIG. 3A. Still alternatively, the grounding source 226 can be provided in another suitable manner. The connection assembly 224 is illustrated in greater detail in FIG. 2B.

FIG. 2B is an enlarged perspective view illustration of a portion of the device body 214, including the earthing therapy device 220 in the form of the conductive surface 220A that is coupled to and/or incorporated within the body cover 222 of the device body 214, and the connection assembly 224, as defined by circle B-B in FIG. 2A. In particular, FIG. 2B illustrates certain details of the connection assembly 224 that is usable for providing the necessary electrical grounding between the earthing therapy device 220 and/or the conductive surface 220A and the grounding source 226.

The connection assembly 224 can be varied. As shown in this embodiment, the connection assembly 224 is provided in the form of a grounding wire 224A, which includes (i) a first end 224F that forms a part of a surface connector 228 for selectively electrically coupling the connection assembly 224 and/or the grounding wire 224A to the earthing therapy device 220 and/or the conductive surface 220A, and (ii) a second end 224S that forms a part of a source connector 230 for selectively electrically coupling the connection assembly 224 and/or the grounding wire 224A to the grounding source 226. Alternatively, the connection assembly 224 can have another suitable design.

As noted, the surface connector 228 is configured to selectively electrically couple the connection assembly 224 and/or the grounding wire 224A to the earthing therapy device 220 and/or the conductive surface 220A. The surface connector 228 can have any suitable design for purposes of providing the selective electrical coupling between the connection assembly 224 and/or the grounding wire 224A and the earthing therapy device 220 and/or the conductive surface 220A. As shown in FIG. 2B, the surface connector 228 includes a first connector member 228A that is connected to the earthing therapy device 220 and/or the conductive surface 220A, and a second connector member 228B that is connected to the grounding wire 224A at or near the first end 224F. In one embodiment, the first connector member 228A is a snap button, and the second connector member 228B is a button insert that is configured to selectively engage the first connector member 228A, the snap button in this particular embodiment. Alternatively, the surface connector 228, the first connector member 228A and/or the second connector member 228B can have another suitable design. For example, in one non-exclusive alternative embodiment, the second member connector 228B can be configured, such as with a snap on a rear surface of the second member connector 228B, to allow another conductive device to also be connected to the ground line that ultimately leads to the grounding source 226.

As further noted, the source connector 230 is configured to selectively electrically couple the connection assembly 224 and/or the grounding wire 224A to the grounding source 226. The source connector 230 can have any suitable design for purposes of providing the selective electrical coupling between the connection assembly 224 and/or the grounding wire 224A and the grounding source 226. As shown in FIG. 2B, the source connector 230 includes a first connector member 230A that is coupled to the grounding source 226, and a second connector member 230B that is connected to the grounding wire 224A at or near the second end 224S. In one embodiment, the first connector member 230A is a power strip, i.e. a ground of the power strip, which is further selectively electrically coupled to the grounding source 226, and the second connector member 230B is a ground adapter that is configured to selectively engage the first connector member 230A, the ground of the power strip in this particular embodiment. Alternatively, the source connector 230, the first connector member 230A and/or the second connector member 230B can have another suitable design. For example, in one non-exclusive alternative embodiment, the second connector member 230B positioned at or near the second end 224S of the grounding wire 224A can be configured to plug directly into the grounding source 226, without the need for a first connector member 230A such as the power strip to effectuate such connection to the grounding source 226.

It is appreciated that any of the components of the connection assembly 224, the surface connector 228, and/or the source connector 230 can be modified from what is shown in FIG. 2B, while still being configured to provide the necessary electrical coupling between earthing therapy device 220 and/or the conductive surface 220A and the grounding source 226. For example, in the embodiment illustrated in FIG. 2A and FIG. 2B, the first connector member 230A, such as in the form of the power strip, is shown as being external to the body of the support body 214 and/or the support device 12 (illustrated in FIG. 1). However, in other embodiments, the first connector member 230A, such as in the form of the power strip, can be provided internally within the overall structure of the support device 12.

FIG. 3A is a simplified perspective view illustration of another embodiment of the device body 314 with the earthing therapy device 320 incorporated therein, and another embodiment of the connection assembly 324 that is configured to be selectively electrically coupled to the earthing therapy device 320. In the embodiment shown in FIG. 3A, the earthing therapy device 320 is again provided in the form of a conductive fabric and/or a conductive surface 320A that is again sewn onto an outer surface 322A of the body cover 322.

The earthing therapy device 320 and/or the conductive surface 320A can be substantially similar in design and function as to what was described in relation to previous embodiments, and the earthing therapy device 320 and/or the conductive surface 320A can be incorporated within the device body 314 in a similar manner as in previous embodiments. Accordingly, the earthing therapy device 320 and/or the conductive surface 320A will not again be described in detail in relation to FIG. 3A.

The connection assembly 324 is again configured to provide desired selective electrically coupling between the earthing therapy device 320 and a grounding source 326. However, in the embodiment illustrated in FIG. 3A, the connection assembly 324 has a different design than what was illustrated in the previous embodiment. In particular, as shown in FIG. 3A, the connection assembly 324 is electrically coupled to and between the earthing therapy device 320 and/or the conductive surface 320A and a body frame 336 of the device body 314. Stated in another manner, in this embodiment, the body frame 336 of the device body 314 is functioning as at least a portion of the conductive path to the grounding source 326. More specifically, in certain embodiments, a power strip (not shown in FIG. 3A) can be incorporated into the overall structure of the device body 314 and/or the support device 12 (illustrated in FIG. 1) for providing at least a portion of the conductive path to the grounding source 326.

As illustrated, the connection assembly 324 is again provided in the form of a grounding wire 324A having a first end 324F that is configured to be selectively electrically coupled to the earthing therapy device 320, and a second end 324S that is configured to be selectively electrically coupled to the grounding source 326. The connection assembly 324 is illustrated in greater detail in FIG. 3B.

FIG. 3B is an enlarged perspective view illustration of a portion of the device body 314, including the earthing therapy device 320 in the form of the conductive fabric or conductive surface 320A that is coupled to and/or incorporated within the body cover 322 of the device body 314, and the connection assembly 324, as defined by circle B-B in FIG. 3A. In particular, FIG. 3B illustrates certain details of the connection assembly 324 that is usable for providing the necessary electrical grounding between the earthing therapy device 320 and/or the conductive surface 320A and the grounding source 326, i.e. the body frame 336 of the device body 314 in this particular embodiment.

In this embodiment, the connection assembly 324 is again provided in the form of a grounding wire 324A, which includes (i) a first end 324F that forms a part of a surface connector 328 for selectively electrically coupling the connection assembly 324 and/or the grounding wire 324A to the earthing therapy device 320 and/or the conductive surface 320A, and (ii) a second end 324S that forms a part of a source connector 330 for selectively electrically coupling the connection assembly 324 and/or the grounding wire 324A to the grounding source 326.

As noted, the surface connector 328 is configured to selectively electrically couple the connection assembly 324 and/or the grounding wire 324A to the earthing therapy device 320 and/or the conductive surface 320A. The surface connector 328 can have any suitable design for purposes of providing the selective electrical coupling between the connection assembly 324 and/or the grounding wire 324A and the earthing therapy device 320 and/or the conductive surface 320A. In this embodiment, as shown in FIG. 3B, the surface connector 328 is substantially similar in design and function as to what was illustrated and described in relation to the embodiment shown in FIG. 2B. More particularly, the surface connector 328 again includes a first connector member 328A that is connected to the earthing therapy device 320 and/or the conductive surface 320A, and a second connector member 328B that is connected to the grounding wire 324A at or near the first end 324F. In one embodiment, the first connector member 328A is again a snap button, and the second connector member 328B is again a button insert that is configured to selectively engage the first connector member 328A, the snap button in this particular embodiment. Alternatively, the surface connector 328, the first connector member 328A and/or the second connector member 328B can have another suitable design.

As further noted, the source connector 330 is configured to selectively electrically couple the connection assembly 324 and/or the grounding wire 324A to the grounding source 326. However, in this embodiment, since a path to the grounding source 326 is provided by the body frame 336 or another portion of the device body 314 (or another portion of the support device 12), the source connector 330 is somewhat different in overall design as compared to previously described embodiments. As shown in FIG. 3B, the source connector 330 includes a first connector member 330A that is coupled to the grounding source 326, and a second connector member 330B that is connected to the grounding wire 324A at or near the second end 324S. In one embodiment, the first connector member 330A is a member receiver that is coupled to and/or incorporated within the body frame 336 or another portion of the device body 314 or the support device 12, and the second connector member 330B is a grounding lug that is configured to be selectively engage and/or be inserted into the first connector member 330A, i.e. the member receiver in this embodiment. Alternatively, the source connector 330, the first connector member 330A and/or the second connector member 330B can have another suitable design.

It is appreciated that any of the components of the connection assembly 324, the surface connector 328, and/or the source connector 330 can be modified from what is shown in FIG. 3B, while still being configured to provide the necessary electrical coupling between earthing therapy device 320 and/or the conductive surface 320A and the grounding source 326.

FIG. 4 is a simplified perspective view illustration of still another embodiment of the device body 414 with the earthing therapy device 420 incorporated therein, and still another embodiment of the connection assembly 424 that is configured to be selectively electrically coupled to the earthing therapy device 420 and the grounding source 426. In the embodiment shown in FIG. 4, the earthing therapy device 420 is again provided in the form of a conductive fabric and/or a conductive surface 420A. However, in this embodiment, the earthing therapy device 420 and/or the conductive surface 420A is instead attached to an inner surface 422B of the body cover 422 of the device body 414. More particularly, in order to ensure that the user can be adequately energized using the earthing therapy device 420, the earthing therapy device 420 and/or the conductive surface 420A can be sewn into or onto the inner surface 422B of the body cover 422 with conductive thread 440.

The conductive thread 440 can be formed from any suitable conductive materials for purposes of enabling the conductive capabilities to pass from the earthing therapy device 420 and/or the conductive surface 420A through the body cover 422 so that the user 10 (illustrated in FIG. 1) positioned on the device body 414 can receive the desired earthing therapy. For example, in certain non-exclusive embodiments, the conductive thread 440 can be formed from one or more of silver, copper, nickel, tin, and stainless steel. Alternatively, the conductive thread 440 can be formed form other suitable conductive materials.

As shown in this embodiment, the connection assembly 424 is substantially similar to the connection assembly 224 illustrated and described in detail in relation to FIG. 2A, and is again configured for providing the necessary electrical grounding between the earthing therapy device 420 and/or the conductive surface 420A and the grounding source 426. More particularly, the connection assembly 424 is again provided in the form of a grounding wire 424A having a first end 424F that is configured to be selectively electrically coupled to the earthing therapy device 420 via a similarly designed surface connector 428, and a second end 424S that is configured to be selectively electrically coupled to the grounding source 426 via a similarly designed source connector 430. As further illustrated in this embodiment, the second end 424S of the grounding wire 424A is again selectively electrically coupled to the grounding source 426, provided in the form of a wall outlet, via a power strip 430A.

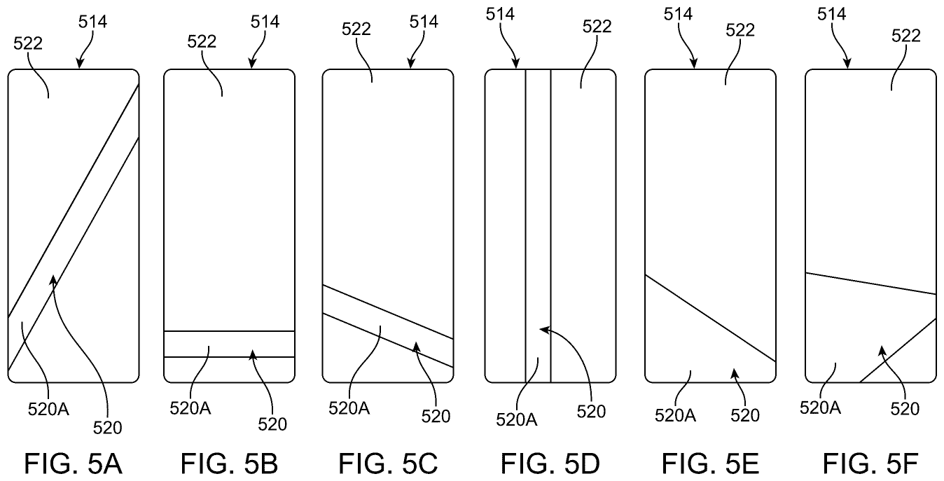

FIGS. 5A-5F are alternative embodiments for the size, shape and positioning of the earthing therapy device 520, such as the conductive fabric and/or conductive surface 520A, as the earthing therapy device 520 is incorporated within a body cover 522 of the device body 514. It is appreciated that the different embodiments shown for the size, shape and positioning of the earthing therapy device 520 and/or the conductive surface 520A as incorporated within the body cover 522 of the device body 514 are simply a representative number of non-exclusive embodiments, and the earthing therapy device 520 can have any suitable size, shape and positioning as the earthing therapy device 520 is incorporated within the body cover 522 of the device body 514.

As described in detail herein in the preceding embodiments, the present invention takes the idea of personal grounding further as it incorporates a conductive fabric and/or a conductive surface into a unit of furniture, a bed, chair, couch, etc. This could be used in hotels, resorts, and spas to provide earthing therapy for clients in treatment rooms and relaxation areas (beds, couches, chairs) or spa environments (massage tables, chairs, loungers, etc.). For example, in the case of beds, the conductive fabric and/or conductive surface could be sewn in the mattress, or mattress cover, and would still be conductive with a thin sheet over it. This would be true for massage table mattresses as well. and is particularly valuable as there is no special sheet required.

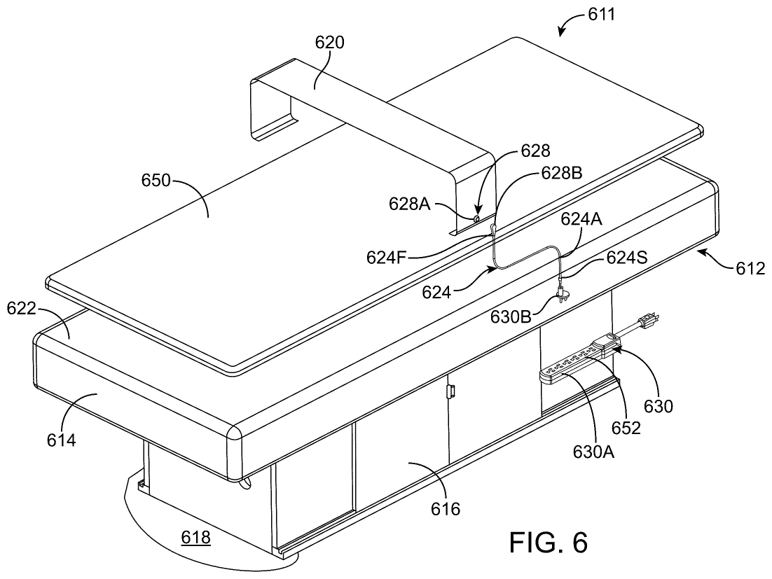

FIG. 6 is a simplified partially exploded perspective view illustration of another embodiment of a support device assembly 611 having features of the present invention. The design of the support device assembly 611 can be varied to suit the needs of a user 10 (illustrated in FIG. 1) of the support device assembly 611. In various embodiments, the support device assembly 611 includes a support device 612, an earthing therapy device 620, and a connection assembly 624 that provides a conductive path and a grounding point for the earthing therapy device 620. Additionally, as shown in this embodiment, the support device assembly 611 can further include a comfort enhancing device 650 (also referred to herein as a “comfort enhancer”), such as a fleece pad or a fleece blanket in certain non-exclusive embodiments, that can be positioned on top of the support device 612 for providing enhanced comfort for the user 10 of the support device assembly 611. As illustrated in FIG. 6, the connection assembly 624 is again configured to provide selective electrical coupling between the earthing therapy device 620 and a grounding source 226 (illustrated in FIG. 2A), such as a wall outlet in certain non-exclusive embodiments. In many embodiments, the conductive path and the grounding point can be provided, at least in part, coupled to and/or within the structure of the support device 612. Alternatively, the support device assembly 611 can include more components or fewer components than those specifically illustrated and described in relation to FIG. 6.

In the particular embodiment shown in FIG. 6, the support device 612 is again a treatment device such as a massage table that supports the user 10 while the user 10 is laying down on the support device 612 while receiving a therapeutic treatment such as a massage. However, it is appreciated that the support device 612 can have another suitable design, and does not have to be provided in the form of a massage table. For example, in one non-exclusive alternative embodiment, the support device 612 can be provided in the form of a massage chair that supports the user 10 in a reclined and/or seated position while the user 10 is receiving a therapeutic treatment such as a massage. In still other embodiments, the support device 612 can be another type of support device, such as a bed, a couch, a chair, a lounger, etc.

The design of the support device 612 can be varied to suit the needs of the user. As illustrated, in certain embodiments, the support device 612 can be substantially similar to the support device 12 illustrated and described in detail in relation to FIG. 1. For example, in the embodiment shown in FIG. 6, the support device 612 again includes (i) a device body 614 that is configured to support the user of the support device 612, and (ii) a support assembly 616 that supports the device body 614 above a surface 618, such as a floor or the ground. Alternatively, the support device 612 can include more components than those specifically illustrated in FIG. 6. For example, in one non-exclusive alternative embodiment, the support device 612 can again further include a headrest assembly (not shown) that is configured to support a face or head of the user 10 when the user 10 is positioned on the device body 614. Such headrest assembly can alternatively be coupled to the device body 614 or integrally formed into the device body 614.

As noted herein, in some embodiments, the device body 614 can be configured to support the user 10 above the surface 618 as the user 10 is receiving a massage or other therapeutic treatment. In other embodiments, the device body 614 can support the user 10 relative to the surface 618 without the need that the user 10 be receiving a therapeutic treatment.

In FIG. 6, the device body 614 is again substantially rectangle-shaped. Alternatively, the device body 614 can have another suitable shape. For example, in certain non-exclusive alternative embodiments, the device body 614 can be substantially oval-shaped, oblong-shaped, rectangular-shaped with one or more rounded corners, or another suitable shape.

In many embodiments, the device body 614 can again include a body frame (not shown), a pad (not shown), and a body cover 622. Alternatively, for example, the device body 614 can be made without the pad and/or without the body cover 622.

The body frame supports the user 10 receiving the massage when the user 10 is positioned on the support device 612. The body frame is generally rigid and can be made of a rigid material such as wood, aluminum, steel, plastic or other suitable materials. In alternative, non-exclusive embodiments, the body frame has a thickness of between approximately five millimeters and 30 millimeters. More particularly, the body frame can have a thickness of approximately 5, 6, 7, 8, 9, 10, 11, 12, 13, 14, 15, 16, 17, 18, 19, 20, 21, 22, 23, 24, 25, 26, 27, 28, 29, or 30 millimeters. However, alternatively, the body frame can have a thickness that is greater than or less than the noted thicknesses.

The pad is secured to and can be positioned substantially on top of the body frame. The pad provides a cushion for the comfort of the user 10 resting on the support device 612. Non-exclusive examples of suitable materials for the pad include foam, memory foam, fleece pads, etc. The pad can have any suitable thickness and any suitable stiffness/resiliency for purposes of comfortably supporting users 10 of many different sizes.

The body cover 622 secures the pad to the body frame and provides a protective covering for the pad. Non-exclusive examples of suitable materials for the body cover 622 include leather, vinyl, plastic, and cloth.

As noted above, the support assembly 616 supports the device body 614 above and/or relative to the surface 618. More particularly, the support assembly 616 extends between the device body 614 and the surface 618 to maintain the device body 614 positioned above and away from the surface 618. It is appreciated that the support assembly 616 can have any suitable design for purposes of supporting the device body 614 above and/or relative to the surface 618. For example, in one embodiment, the support assembly 616 can include one or more legs and/or feet that are fairly lightweight, but still provide desired stability for the support device 612. Alternatively, the support assembly 616 can have another suitable design.

As with the previous embodiments, the support assembly 616 can again be adjustable so as to enable a height of the device body 614 relative to the surface 618 to be selectively adjusted. It is again appreciated that any such height adjustment can be accomplished in any suitable manual or automated manner.

In certain embodiments, the support assembly 616 can again be configured to provide one or more storage compartments, e.g., drawers, that are usable for storage of materials that can be used during any therapeutic treatments.

As noted above, in certain implementations, the user 10 may want to enhance their comfort when they are positioned on the support device 612 through use of the comfort enhancer 650 that can be positioned on top of the support device 612. In certain embodiments, the comfort enhancer 650 can be provided in the form of a fleece pad or a fleece blanket, which can be selectively positioned on top of the device body 614.

The earthing therapy device 620 can be any device suitable for providing grounding or earthing therapy to the user 10 of the support device assembly 611. In certain embodiments, as shown, the earthing therapy device 620 can be provided in the form of a conductive band that can be positioned at least partially about the comfort enhancer 650, such as the fleece pad, as the comfort enhancer 650 is mounted on top of the device body 614. As illustrated, it is appreciated that the earthing therapy device 620, such as the conductive band, can help to provide at least a portion of the conductive path and/or the grounding point between the user 10 and the grounding source 226, as well as functioning to effectively couple the comfort enhancer 650 to the device body 614 of the support device 612.

The earthing therapy device 620 in the form of the conductive band can be formed from any suitable conductive materials. For example, in certain non-exclusive alternative embodiments, the earthing therapy device 620, i.e. the conductive band, can be formed from one or more of conductive leather, conductive vinyl, conductive plastic, and conductive cloth, which can be similar to the materials used for the body cover 622, with the exception of including conductive properties. Alternatively, the earthing therapy device 620, i.e. the conductive band, can be formed from other suitable conductive materials.

The connection assembly 624 is usable for further providing the necessary electrical grounding between the earthing therapy device 620 and the grounding source 226. In particular, in this embodiment, the connection assembly 624 is again configured to be electrically coupled to and between the earthing therapy device 620 and the grounding source 226. More specifically, as shown, a first end 624F of the connection assembly 624 can be electrically coupled to the earthing therapy device 620, provided in the form of the conductive band; and a second end 624S of the connection assembly 624 can be electrically coupled to the grounding source 226, such as by being electrically coupled to a power strip 652, which can in turn be electrically coupled to the grounding source 226, such as the wall outlet, or another type of ground. As such, the connection assembly 624 provides at least a portion of the desired electrical coupling between the earthing therapy device 620, as mounted on and/or coupled to the support device 612, such as a massage table or a massage chair, and the grounding source 226.

In some embodiments, as illustrated, the power strip 652 can be mounted onto and/or incorporated into the overall structure of the device body 614. With such design, potentially tripping hazards near or about the support device 612 can be limited. It is appreciated that the power strip 652 is illustrated in FIG. 6 as being positioned externally relative to the support device 612. However, it is further appreciated that the power strip 652, or another comparable grounding component, can alternatively be positioned internally within the structure of the support device 612.

In certain alternative embodiments, the grounding source 226 itself can be incorporated into the device body 614 in any suitable manner, thus eliminating the need to run external cables to the grounding source 226. Thus, the present invention can provide an elegant solution to grounding a user 10 who is resting on the support device 612 that is covered with the comfort enhancer 650, such as a fleece pad or fleece blanket.

The design of the connection assembly 624 can be varied. As shown in this embodiment, the connection assembly 624 is provided in the form of a grounding wire 624A, which includes (i) the first end 624F that forms a part of a surface connector 628 for selectively electrically coupling the connection assembly 624 and/or the grounding wire 624A to the earthing therapy device 620, i.e. the conductive band, and (ii) the second end 624S that forms a part of a source connector 630 for selectively electrically coupling the connection assembly 624 and/or the grounding wire 624A to the grounding source 226. Alternatively, the connection assembly 624 can have another suitable design.

The surface connector 628 can have any suitable design for purposes of providing the selective electrical coupling between the connection assembly 624 and/or the grounding wire 624A and the earthing therapy device 620. As shown in FIG. 6, the surface connector 628 includes a first connector member 628A that is connected to the earthing therapy device 620, and a second connector member 628B that is connected to the grounding wire 624A at or near the first end 624F. In one embodiment, the first connector member 628A is a snap button, and the second connector member 628B is a button insert that is configured to selectively engage the first connector member 628A, the snap button in this particular embodiment. Alternatively, the surface connector 628, the first connector member 628A and/or the second connector member 628B can have another suitable design.

The source connector 630 can have any suitable design for purposes of providing the selective electrical coupling between the connection assembly 624 and/or the grounding wire 624A and the grounding source 626. As shown in FIG. 6, the source connector 630 includes a first connector member 630A that is coupled to the grounding source 226, and a second connector member 630B that is connected to the grounding wire 624A at or near the second end 624S. In one embodiment, the first connector member 630A is a power strip, i.e. a ground of the power strip 652, which is further selectively electrically coupled to the grounding source 226, and the second connector member 630B is a ground adapter that is configured to selectively engage the first connector member 630A, the ground of the power strip 652 in this particular embodiment. Alternatively, the source connector 630, the first connector member 630A and/or the second connector member 630B can have another suitable design.

It is appreciated that any of the components of the connection assembly 624, the surface connector 628, and/or the source connector 630 can be modified from what is shown in FIG. 6, while still being configured to provide the necessary electrical coupling between earthing therapy device 620 and the grounding source 226.

As so described, the typical surface of a support device 612, such as a massage table, is non-conductive. Often, more non-conductive layers, such as the comfort enhancer 650, are placed on top of the support device 612 for increased comfort and/or warmth. By placing the earthing therapy device 620, such as a conductive band, in touch with the client, the earthing therapy device 620 can utilize the grounding attachment points provided to allow said device to function for the purpose of earthing therapy.

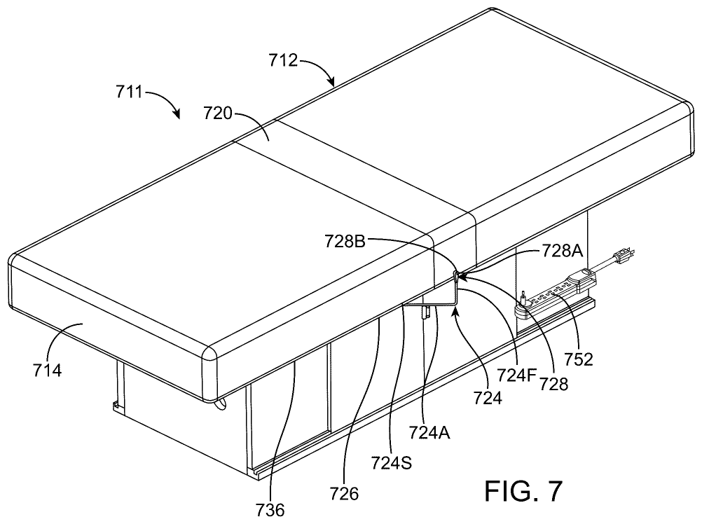

FIG. 7 is a perspective view illustration of a portion of another embodiment of the support device assembly 711 having features of the present invention. In this embodiment, the support device assembly 711 encompasses a support device 712, such as a massage table, with its own built-in ground attachment, and an earthing therapy device 720, in the form of a conductive band similar to what has been illustrated and described in relation to FIG. 6, that is positioned about and/or coupled to at least a portion of the device body 714 of the support device. As shown, the support device 712 is similar in design and functionality to what has been illustrated and described in relation to FIG. 6. However, a separate comfort enhancer, such as comfort enhancer 650 (illustrated in FIG. 6), is not included in this embodiment.

As illustrated, the earthing therapy device 720 is positioned about at least a portion of the device body 714 to help provide at least a portion of the conductive path and/or the grounding point between the user 10 (illustrated in FIG. 1) who is positioned on the device body 714 and a grounding source 726.

In this embodiment, the connection assembly 724 is again configured to provide desired selective electrically coupling between the earthing therapy device 720 and the grounding source 726. More particularly, similar to the embodiment illustrated and described in relation to FIGS. 3A and 3B, the connection assembly 724 is again electrically coupled to and between the earthing therapy device 720 and a body frame 736 of the device body 714. Stated in another manner, in this embodiment, the body frame 736 of the device body 714 is functioning as at least a portion of the conductive path to the grounding source 726. More specifically, in certain embodiments, a power strip 752 can be incorporated into the overall structure of the device body 714 and/or the support device 712 for providing at least a portion of the conductive path to the grounding source 726. As above, it is appreciated that the power strip 752 can be provided either externally or internally relative to the overall structure of the device body 714 and/or the support device 712.

In this embodiment, the connection assembly 724 is again provided in the form of a grounding wire 724A having a first end 724F that forms a part of a surface connector 728 for selectively electrically coupling the connection assembly 724 to the earthing therapy device 720, and a second end 724S that forms a part of a source connector (not shown) for selectively electrically coupling the connection assembly 724 to the grounding source 726, such as via the body frame 736, the power strip 752, or another suitable grounding component incorporated within the support device 712 in this particular embodiment.

The surface connector 728 can have any suitable design for purposes of providing the selective electrical coupling between the connection assembly 724 and/or the grounding wire 724A and the earthing therapy device 620. In this embodiment, as shown in FIG. 7, the surface connector 728 is substantially similar in design and function as to what was illustrated and described in relation to the embodiment shown in FIG. 6. More particularly, the surface connector 728 again includes a first connector member 728A that is connected to the earthing therapy device 720, such as in the form of the conductive band, and a second connector member 728B that is connected to the grounding wire 724A at or near the first end 724F. In one embodiment, the first connector member 728A is again a snap button, and the second connector member 728B is again a button insert that is configured to selectively engage the first connector member, the snap button in this particular embodiment. Alternatively, the surface connector 728, the first connector member 728A and/or the second connector member 728B can have another suitable design.

The source connector can also have any suitable design for purposes of providing the selective electrical coupling between the connection assembly 724 and/or the grounding wire 724A and the grounding source 726. Similar to the embodiment shown in FIGS. 3A and 3B, the source connector includes a first connector member (not shown) that is coupled to the grounding source 726, and a second connector member (not shown) that is connected to the grounding wire 724A at or near the second end 724S. In one embodiment, the first connector member is a member receiver that is coupled to and/or incorporated within the body frame 736 or another portion of the device body 714 or the support device 712, and the second connector member is a grounding lug that is configured to be selectively engage and/or be inserted into the first connector member, i.e. the member receiver in this embodiment. Alternatively, the source connector, the first connector member and/or the second connector member can have another suitable design.

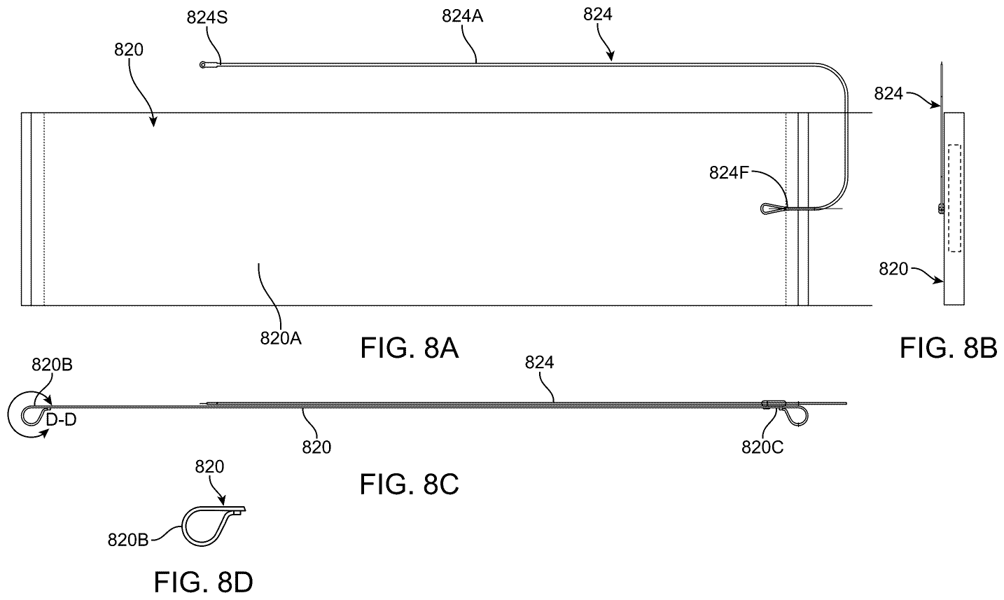

FIG. 8A is a simplified top view illustration of an embodiment of the earthing therapy device 820 and a portion of an embodiment of the connection assembly 824 illustrated in FIG. 6. As noted above, the earthing therapy device 820 can be positioned on top of the comfort enhancer 650 (illustrated in FIG. 6), such as the fleece pad and/or fleece blanket, which can be selectively positioned on top of the device body 614 (illustrated in FIG. 6) of the support device 612 (illustrated in FIG. 6). The earthing therapy device 820 is again configured to provide a source of conductive energy for purposes of providing earthing therapy to the user 10 (illustrated in FIG. 1) who is positioned on top of the support device 612. As also noted above, the earthing therapy device 820 is further usable for helping to couple the comfort enhancer 650, which is non-conductive, on top of the device body 614.

It is appreciated that the earthing therapy device 820, such as the conductive band, can be of any suitable size, which can be the same size as the entire upper surface of the device body 614 or any smaller portion thereof.

As further shown in FIG. 8A, the connection assembly 824 can be electrically coupled to the conductive surface 820A of the earthing therapy device 820 in order to provide the desired conductive path and a grounding point for the earthing therapy device 820.

The connection assembly 824 can have a design that is similar to what has been illustrated and described in relation to previous embodiments. For example, in some embodiments, the connection assembly 824 can again be provided in the form of a grounding wire 824A, including a first end 824F that can be selectively electrically coupled to the conductive surface 820A of the earthing therapy device 820 in any suitable manner, and a second end 824S that can be selectively electrically coupled to a grounding source (such as, for example, the grounding source 226 illustrated in FIG. 2A) in any suitable manner.

FIG. 8B is a simplified end view illustration of the earthing therapy device 820 and the connection assembly 824 illustrated in FIG. 8A.

FIG. 8C is a simplified side view illustration of the earthing therapy device 820 and the connection assembly 824 illustrated in FIG. 8A. As shown in FIG. 8C, opposing ends 820B, 820C of the earthing therapy device 820, i.e. the conductive band, can have a looped design for purposes of receiving a weighted object therewithin so that the earthing therapy device 820 can remain in a desired position relative to the device body 614 (illustrated in FIG. 6) and/or the comfort enhancer 650 (illustrated in FIG. 6) without the earthing therapy device 820 being specifically attached to the device body 614. In some non-exclusive embodiments, a dowel, which can be made from any suitable metals or wood, can be positioned within the looped opposing ends 820B, 820C of the earthing therapy device 820 to provide the desired weighting at either end 820B, 820C of the earthing therapy device 820.

FIG. 8D is an enlarged schematic view illustration of a portion of the earthing therapy device 820 as defined by circle D-D in FIG. 8C. In particular, FIG. 8D illustrates the looped design at one end 820B of the earthing therapy device 820.

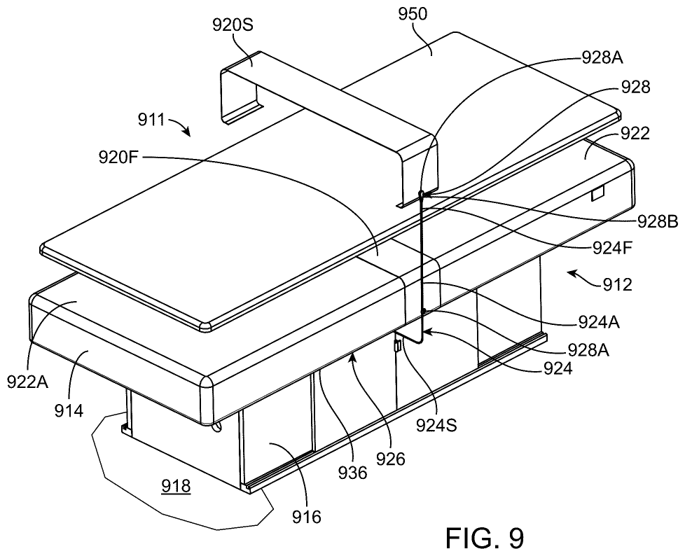

FIG. 9 is a simplified perspective view illustration of a portion of still yet another embodiment of the support device assembly 911 having features of the present invention. More particularly, as shown, FIG. 9 is a simplified perspective view illustration of the support device assembly 911 that incorporates features of the previous embodiments illustrated and described in relation to FIGS. 2A-2B, and FIG. 6.

The design of the support device assembly 911 can be varied to suit the needs of a user 10 (illustrated in FIG. 1) of the support device assembly 911. In various embodiments, the support device assembly 911 includes a support device 912, a first earthing therapy device 920F, a comfort enhancer 950, a second earthing therapy device 920S, and a connection assembly 924 that provides a conductive path and a grounding point for one or both of the first earthing therapy device 920F and the second earthing therapy device 920S. Similar to the embodiment illustrated in FIG. 6, the comfort enhancer 950, such as a fleece pad or a fleece blanket in certain non- exclusive embodiments, can be positioned on top of the support device 912 for providing enhanced comfort for the user 10 of the support device assembly 911. Alternatively, the support device assembly 911 can include more components or fewer components than those specifically illustrated and described in relation to FIG. 9.

In the particular embodiment shown in FIG. 9, the support device 912 is again a treatment device such as a massage table that supports the user 10 while the user 10 is laying down on the support device 912 while receiving a therapeutic treatment such as a massage. However, it is appreciated that the support device 912 can have another suitable design, and does not have to be provided in the form of a massage table. For example, in one non-exclusive alternative embodiment, the support device 912 can be provided in the form of a massage chair that supports the user 10 in a reclined and/or seated position while the user 10 is receiving a therapeutic treatment such as a massage. In still other embodiments, the support device 912 can be another type of support device, such as a bed, a couch, a chair, a lounger, etc.

The design of the support device 912 can be varied to suit the needs of the user. As illustrated, in certain embodiments, the support device 912 can again be substantially similar to the support device 12 illustrated and described in detail in relation to FIG. 1 and/or the support device 612 illustrated and described in detail in relation to FIG. 6. For example, in the embodiment shown in FIG. 9, the support device 912 again includes (i) a device body 914 that is configured to support the user of the support device 912, and (ii) a support assembly 916 that supports the device body 914 above a surface 918, such as a floor or the ground. Alternatively, the support device 912 can include more components than those specifically illustrated in FIG. 9. For example, in one non-exclusive alternative embodiment, the support device 912 can again further include a headrest assembly (not shown) that is configured to support a face or head of the user 10 when the user 10 is positioned on the device body 914. Such headrest assembly can alternatively be coupled to the device body 914 or integrally formed into the device body 914.

As noted herein, in some embodiments, the device body 914 can be configured to support the user 10 above the surface 918 as the user 10 is receiving a massage or other therapeutic treatment. In other embodiments, the device body 914 can support the user 10 relative to the surface 918 without the need that the user 10 be receiving a therapeutic treatment.

In FIG. 9, the device body 914 is again substantially rectangle-shaped. Alternatively, the device body 914 can have another suitable shape. For example, in certain non-exclusive alternative embodiments, the device body 914 can be substantially oval-shaped, oblong-shaped, rectangular-shaped with one or more rounded corners, or another suitable shape.

In many embodiments, the device body 914 can again include a body frame 936, a pad (not shown), and a body cover 922. Alternatively, for example, the device body 914 can be made without the pad and/or without the body cover 922.

The body frame 936 supports the user 10 receiving the massage when the user 10 is positioned on the support device 912. The body frame 936 is generally rigid and can be made of a rigid material such as wood, aluminum, steel, plastic or other suitable materials. In alternative, non-exclusive embodiments, the body frame 936 has a thickness of between approximately five millimeters and 30 millimeters. More particularly, the body frame 936 can have a thickness of approximately 5, 6, 7, 8, 9, 10, 11, 12, 13, 14, 15, 16, 17, 18, 19, 20, 21, 22, 23, 24, 25, 26, 27, 28, 29, or 30 millimeters. However, alternatively, the body frame 936 can have a thickness that is greater than or less than the noted thicknesses.

The pad is secured to and can be positioned substantially on top of the body frame 936. The pad provides a cushion for the comfort of the user 10 resting on the support device 912. Non-exclusive examples of suitable materials for the pad include foam, memory foam, fleece pads, etc. The pad can have any suitable thickness and any suitable stiffness/resiliency for purposes of comfortably supporting users 10 of many different sizes.

The body cover 922 secures the pad to the body frame and provides a protective covering for the pad. Non-exclusive examples of suitable materials for the body cover 922 include leather, vinyl, plastic, and cloth.

As noted above, the support assembly 916 supports the device body 914 above and/or relative to the surface 918. More particularly, the support assembly 916 extends between the device body 914 and the surface 918 to maintain the device body 914 positioned above and away from the surface 918. It is appreciated that the support assembly 916 can have any suitable design for purposes of supporting the device body 914 above and/or relative to the surface 918. For example, in one embodiment, the support assembly 916 can include one or more legs and/or feet that are fairly lightweight, but still provide desired stability for the support device 912. Alternatively, the support assembly 916 can have another suitable design.

As with the previous embodiments, the support assembly 916 can again be adjustable so as to enable a height of the device body 914 relative to the surface 918 to be selectively adjusted. It is again appreciated that any such height adjustment can be accomplished in any suitable manual or automated manner.

In certain embodiments, the support assembly 916 can again be configured to provide one or more storage compartments, e.g., drawers, that are usable for storage of materials that can be used during any therapeutic treatments.

Similar to the embodiment illustrated in FIGS. 2A and 2B, as shown in FIG. 9, the first earthing therapy device 920F can encompass a conductive fabric or material that can be incorporated into the body cover 922 of the device body 914. As with previous embodiments, the first earthing therapy device 920F can be incorporated into the body cover 922 of the device body 914 in any suitable manner and in any suitable position. For example, in one embodiment, the first earthing therapy device 920F can be sewn onto or otherwise attached to an outer surface 922A of the body cover 922. In another embodiment, the first earthing therapy device 920F can be sewn into an inner surface (not shown) or the underside of the body cover 922 using conductive thread, such as shown in FIG. 4.

In certain implementations, the user 10 may want to enhance their comfort when they are positioned on the support device 912 through use of the comfort enhancer 950 that can be positioned on top of the support device 912 and/or the device body 914. However, when utilized, the comfort enhancer 950 would typically be formed from an insulating material that would inhibit the conductive properties of the first earthing therapy device 920F from being transmitted to the user 10. Thus, in such implementations, the support device assembly 911 can further incorporate and utilize the second earthing therapy device 920S.

The second earthing therapy device 920S can be any device suitable for providing grounding or earthing therapy to the user 10 of the support device assembly 911, as the user 10 is positioned on the comfort enhancer 950 which is positioned on the device body 914. In certain embodiments, similar to what has been illustrated and described in relation to FIG. 6, the second earthing therapy device 920S can be provided in the form of a conductive band that can be positioned at least partially about the comfort enhancer 950, such as the fleece pad, as the comfort enhancer 950 is mounted on top of the device body 914. As illustrated, it is appreciated that the second earthing therapy device 920S, such as the conductive band, can help to provide at least a portion of the conductive path and/or the grounding point between the user 10 and a grounding source 926, as well as functioning to effectively couple the comfort enhancer 950 to the device body 914 of the support device 912.

The connection assembly 924 is usable for further providing the necessary electrical grounding between the first earthing therapy device 920F and/or the second earthing therapy device 920S and the grounding source 926. In particular, in this embodiment, the connection assembly 924 can again be provided in the form of a grounding wire 924A that is again configured to be electrically coupled to and between the first earthing therapy device 920F and/or the second earthing therapy device 920S and the grounding source 926. More specifically, as shown, a first end 924F of the grounding wire 924A can be electrically coupled to one or both of the first earthing therapy device 920F and the second earthing therapy device 920S; and a second end 924S of the grounding wire 924A can be electrically coupled to the grounding source 926, with an appropriate conductive path being potentially incorporated, at least in part, within the body frame 936 of the device body 914 or other portions of the support device 912. As such, the connection assembly 924 provides at least a portion of the desired electrical coupling between one or both of the first earthing therapy device 920F and the second earthing therapy device 920S, as incorporated into and/or coupled to the support device 912, such as a massage table or a massage chair, and the grounding source 926.

The design of the connection assembly 924 can be varied. As noted, in the embodiment shown in FIG. 9, the connection assembly 924 is provided in the form of the grounding wire 924A, which includes (i) the first end 924F that forms a part of a surface connector 928 for selectively electrically coupling the connection assembly 924 and/or the grounding wire 924A to the first earthing therapy device 920F and/or the second earthing therapy device 920S, and (ii) the second end 924S that forms a part of a source connector (not shown) for selectively electrically coupling the connection assembly 924 and/or the grounding wire 924A to the grounding source 926. Alternatively, the connection assembly 924 can have another suitable design.

The surface connector 928 can have any suitable design for purposes of providing the selective electrical coupling between the connection assembly 924 and/or the grounding wire 924A and the first earthing therapy device 920F and/or the second earthing therapy device 920S. As shown in FIG. 9, the surface connector 928 again includes a first connector member 928A and a second connector member 928B that are similar to previous embodiments, with the first connector member 928A being connected to the earthing therapy device 920F, 920S, and the second connector member 928B being connected to the grounding wire 924A at or near the first end 924F. However, in this embodiment, due to the inclusion of both the first earthing therapy device 920F and the second earthing therapy device 920S, each of the first earthing therapy device 920F and the second earthing therapy device 920S can have a first connector member 928A that is connected thereto. Additionally, when both earthing therapy devices 920F, 920S are included, the first connector member 928A connected to the second earthing therapy device 920S can further be configured and positioned to engage the first connector member 928A connected to the first earthing therapy device 920F. Accordingly, the desired conductive path can be provided to both the first earthing therapy device 920F and the second earthing therapy device 920S.

In one embodiment, the first connector member 928A is a snap button, and the second connector member 928B is a button insert that is configured to selectively engage the first connector member 928A, the snap button in this particular embodiment. Alternatively, the surface connector 928, the first connector member 928A and/or the second connector member 928B can have another suitable design.

The source connector can have any suitable design for purposes of providing the selective electrical coupling between the connection assembly 924 and/or the grounding wire 924A and the grounding source 926. In the embodiment shown in FIG. 9, the source connector can have a design that is substantially similar to what was illustrated and described in relation to FIGS. 3A and 3B and/or in FIG. 7. For example, in one embodiment, the source connector includes a first connector member (not shown) that is coupled to the grounding source 926, and a second connector member (not shown) that is connected to the grounding wire 924A at or near the second end 924S. In one embodiment, the first connector member is a member receiver that is coupled to and/or incorporated within the body frame 936 or another portion of the device body 914 or the support device 912, and the second connector member is a grounding lug that is configured to be selectively engage and/or be inserted into the first connector member, i.e. the member receiver in this embodiment. Alternatively, the source connector, the first connector member and/or the second connector member can have another suitable design.

It is appreciated that any of the components of the connection assembly 924, the surface connector 928, and/or the source connector can be modified from what is shown in FIG. 9, while still being configured to provide the necessary electrical coupling between earthing therapy device 920 and the grounding source 926.

As provided herein, the present invention allows for use of earthing therapy in a massage environment, while giving the therapist more freedom of movement and preventing a possible trip hazard from external cords.

It is understood that although a number of different embodiments of the support device assembly and/or the support device have been illustrated and described herein, one or more features of any one embodiment can be combined with one or more features of one or more of the other embodiments, provided that such combination satisfies the intent of the present invention.

While a number of exemplary aspects and embodiments of the support device assembly and/or the support device have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A support device assembly for supporting a user relative to a surface, the support device assembly comprising:
a support device that supports the user relative to the surface, the support device including a device body; and
an earthing therapy device that is configured to provide grounding therapy to the user as the user is positioned on the support device, the earthing therapy device being one of (i) incorporated into, and (ii) coupled to the device body.

2. The support device assembly of claim 1 wherein the earthing therapy device includes a conductive surface that is formed from at least one of conductive leather, conductive vinyl, conductive plastic, and conductive cloth.

3. The support device assembly of claim 1 wherein the earthing therapy device includes a conductive surface that is incorporated into the device body.

4. The support device assembly of claim 1 wherein the earthing therapy device includes a conductive surface that is coupled to the device body.

5. The support device assembly of claim 1 wherein the device body includes a body cover; and wherein the earthing therapy device is one of incorporated into and coupled to the body cover.

6. The support device assembly of claim 5 wherein the earthing therapy device is one of incorporated into and coupled to an outer surface of the body cover.

7. The support device assembly of claim 5 wherein the earthing therapy device is one of incorporated into and coupled to an inner surface of the body cover.

8. The support device assembly of claim 7 wherein the earthing therapy device is coupled to the inner surface of the body cover using conductive thread.

9. The support device assembly of claim 8 wherein the conductive thread is formed from at least one of silver, copper, nickel, tin, and stainless steel.

10. The support device assembly of claim 1 further comprising a comfort enhancer that is configured to be selectively positioned adjacent to the device body; and wherein the earthing therapy device includes a conductive surface that is positioned at least partially about the comfort enhancer.

11. The support device assembly of claim 10 wherein the earthing therapy device is configured to couple the comfort enhancer to the device body.

12. The support device assembly of claim 1 further comprising a connection assembly that selectively electrically couples the earthing therapy device to a grounding source.

13. The support device assembly of claim 12 wherein the connection assembly includes a grounding wire having a first end that is selectively electrically coupled to the earthing therapy device, and a second end that is selectively electrically coupled to the grounding source.

14. The support device assembly of claim 13 wherein the first end of the grounding wire is selectively electrically coupled to the earthing therapy device via a surface connector, the surface connector including a first connector member that is connected to the earthing therapy device, and a second connector member that is connected to the grounding wire near the first end of the grounding wire, the second connector member being configured to selectively engage the first connector member.

15. The support device assembly of claim 13 wherein the second end of the grounding wire is selectively electrically coupled to the grounding source via a source connector, the source connector including a first connector member that is connected to the grounding source, and a second connector member that is connected to the grounding wire near the second end of the grounding wire, the second connector member being configured to selectively engage the first connector member.

16. The support device assembly of claim 15 wherein the grounding source includes a wall outlet.

17. The support device assembly of claim 16 wherein the first connector member includes a power strip that is selectively electrically coupled to the grounding source, and the second connector member includes a ground adapter that is connected to the grounding wire near the second end of the grounding wire, the ground adapter being configured to selectively electrically engage the power strip.

18. The support device assembly of claim 16 wherein the first connector member is incorporated within the device body.

19. The support device assembly of claim 15 wherein the device body of the support device further includes a body frame; and wherein the first connector member includes a member receiver that is incorporated within the body frame, and the second connector member includes a grounding lug that is configured to selectively engage the member receiver.

20. The support device assembly of claim 1 wherein the support device is a treatment device that supports the user relative to the surface as the user is receiving a therapeutic treatment.
